(19) United States
(12) Patent Application Publication
(10) Pub. No.: US 2026/0146209 A1
(43) Pub. Date: May 28, 2026

(54) TECHNIQUES FOR METHANE AND ORGANIC WASTE CONVERSION

(71) Applicant: Circumvent Global Inc., Baltimore, MD (US)

(72) Inventors: Bruce LICHOROWIC, Baltimore, MD (US); David SAUNDERS, Baltimore, MD (US)

(21) Appl. No.: 19/401,782

(22) Filed: Nov. 26, 2025

Related U.S. Application Data
(60) Provisional application No. 63/724,940, filed on Nov. 26, 2024.

(51) Int. Cl. C10J 3/78; C01B 3/02; C07C 1/04; C10G 2/00; C10L 1/04

(52) U.S. Cl. CPC C10J 3/78; C01B 3/02; C07C 1/0485; C10G 2/34; C10L 1/04; C10G 2300/1003; C10G 2300/1018; C10G 2300/1081; C10G 2400/04; C10J 2300/0906; C10J 2300/0909; C10J 2300/0916; C10J 2300/0946; C10J 2300/0956; C10J 2300/1659; C10L 2290/04; C10L 2290/08; C10L 2290/28; C10L 2290/42

(57) ABSTRACT

Techniques are described to capture methane emissions from barns or other agricultural sites and to provide both the methane and a waste feedstock to a gasifier. The waste feedstock may comprise both organic agricultural waste (e.g., manure, rejected feed) and synthetic waste (e.g., plastic wrappers or feed bags, wood), which may be combined before being provided to a gasification process. The gasifier may convert the waste feedstock and methane gas into syngas, which, for example, may be fed into a boiler to produce steam, or may be used to produce fuel through a Fischer-Tropsch process.

### CROSS-REFERENCE TO RELATED APPLICATIONS

The present application claims the benefit under 35 U.S.C. § 119(e) of U.S. Provisional Patent Application No. 63/724,940 , filed Nov. 26, 2024, titled “SYSTEM AND METHOD FOR METHANE AND ORGANIC WASTE CONVERSION ON DAIRY FARMS AND LANDFILLS,” which is hereby incorporated by reference in its entirety.

### BACKGROUND

Methane emissions and organic waste management pose substantial environmental and operational challenges for dairy farms and landfills. On dairy farms, methane is produced as a byproduct of livestock digestion, while the accumulation of plastic, wood, and organic waste materials contributes to pollution and inefficient waste disposal. Similarly, landfills generate significant amounts of methane during the anaerobic decomposition of organic waste, further exacerbating greenhouse gas emissions and their associated climate impacts.

### SUMMARY

According to some aspects, the techniques described herein relate to a method including: directing methane gas and a waste feedstock into a gasifier, wherein the waste feedstock includes organic agricultural waste and synthetic waste; and heating the methane gas and the waste feedstock in the gasifier to produce syngas.

According to some aspects, the techniques described herein relate to a system including: a feed pipeline system configured to convey methane gas and a waste feedstock, wherein the waste feedstock includes organic agricultural waste and synthetic waste; and a gasifier configured to receive the methane gas and the waste feedstock from the feed pipeline system and to heat the methane gas and the waste feedstock to produce syngas.

The foregoing apparatus and method embodiments may be implemented with any suitable combination of aspects, features, and acts described above or in further detail below. These and other aspects, embodiments, and features of the present teachings can be more fully understood from the following description in conjunction with the accompanying drawings.

### BRIEF DESCRIPTION OF DRAWINGS

Various aspects and embodiments will be described with reference to the following figures. It should be appreciated that the figures are not necessarily drawn to scale. In the drawings, each identical or nearly identical component that is illustrated in various figures is represented by a like numeral. For purposes of clarity, not every component may be labeled in every drawing.

FIG. 1 is a schematic drawing of a system suitable for practicing aspects of the present disclosure, according to some embodiments;

FIG. 2 depicts an illustrative example of a system suitable for utilizing syngas in a Fischer-Tropsch reactor, according to some embodiments; and

FIG. 3 is a flowchart of a methane and waste conversion method, according to some embodiments.

### DETAILED DESCRIPTION

Some methods for addressing methane emissions and organic waste are often plagued by inefficiencies, high costs, and wasteful practices. These limitations create an urgent need for innovative systems capable of simultaneously mitigating emissions, managing waste streams, and producing renewable energy.

This application discloses techniques to capture methane emissions from barns or other agricultural sites and to provide both the methane and a waste feedstock to a gasifier. The waste feedstock may comprise both organic agricultural waste (e.g., manure, rejected feed) and synthetic waste (e.g., plastic wrappers or feed bags, wood), which may be combined before being provided to a gasification process. The gasifier may convert the waste feedstock and methane gas into syngas, which, for example, may be fed into a boiler to produce steam, or may be used to produce fuel through a Fischer-Tropsch process. In some embodiments, steam produced by the gasifier may power an electric generator for on-farm use and/or for export to the power grid. Alternatively, the steam can be used directly for heating purposes.

The techniques described herein may provide one or more of the following benefits: (a) Methane Mitigation: Reduces harmful greenhouse gas emissions by capturing methane directly from barns and landfills; (b) Waste Repurposing: Converts plastic, wood, and organic waste into valuable energy resources; and/or (c) Renewable Energy Production: Produces clean energy for on-site use or sale, enhancing farm or landfill sustainability and economic resilience.

This application discloses embodiments in which a gasifier is fueled by minimal natural gas or other suitable feedstock typically available at production sites. In such cases, an external cooling apparatus may optionally be included and configured to assist in drawing cooler air or water to the gasifier for cooling purposes.

In some embodiments, a system includes a methane capture unit designed for flexible deployment. The methane capture unit can be positioned in barns or landfill gas collection zones to capture methane emissions and can be equipped with pipelines to transfer the collected methane directly to a gasifier for processing. The gasifier can serves as the system's core, processing a diverse range of organic waste materials, including plastics, wood, and animal waste, alongside captured methane. Operating at high temperatures, the gasifier breaks down these feedstock materials into syngas, a mixture of hydrogen, carbon monoxide, and methane. In one example, syngas can be produced and can become a versatile energy source for further applications.

In some embodiments, the syngas generated by the gasifier is burned to power a boiler system designed to produce steam. The steam output is directed either to heating systems for thermal applications or to an electric generator that converts the energy into electricity. The generated electricity can be utilized for farm operations or distributed to the power grid. To optimize the system's performance, a control system can manage the flow of methane, organic materials, and syngas, ensuring maximum energy efficiency. The control system may also monitor emissions and adjusts operating parameters to maintain compliance with environmental regulations.

The techniques described herein can integrate waste management, methane mitigation, and energy production into a unified system. By reducing methane emissions, converting waste into usable energy, and generating additional revenue through electricity export, the techniques and their relates systems and method offer environmental and/or economic advantages. The gasification of farm waste and the utilization of animal emissions not only mitigate methane release but also harness valuable gases for energy production. By combining these processes, the system reduces reliance on standard fuels, enhances efficiency, and minimizes overall emissions.

Following below are more detailed descriptions of various concepts related to, and embodiments of, techniques for producing syngas from a combination of methane gas and waste feedstock. It should be appreciated that various aspects described herein may be implemented in any of numerous ways. Examples of specific implementations are provided herein for illustrative purposes only. In addition, the various aspects described in the embodiments below may be used alone or in any combination, and are not limited to the combinations explicitly described herein.

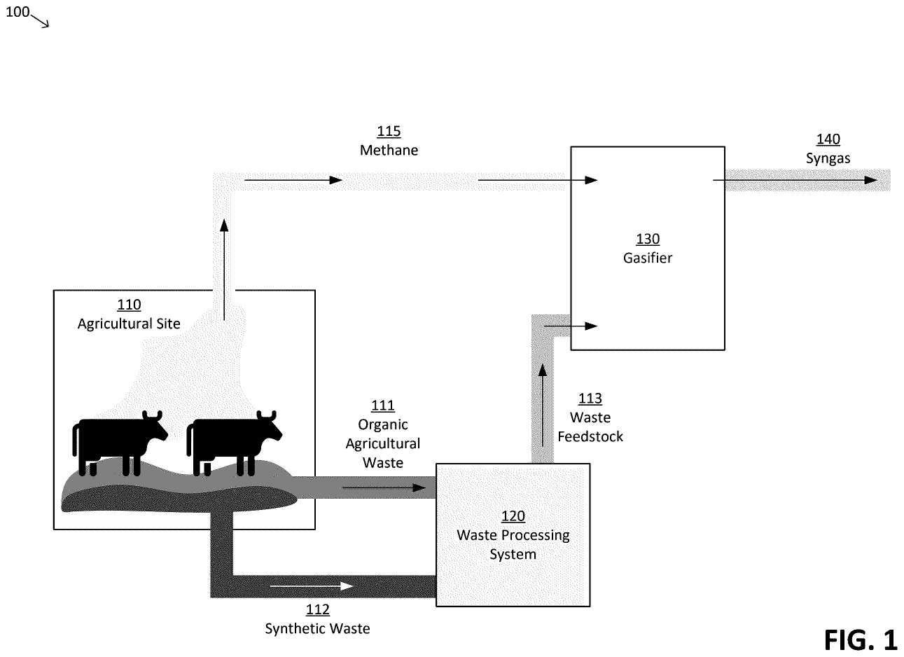

FIG. 1 is a schematic drawing of a system suitable for practicing aspects of the present disclosure, according to some embodiments. In the example of FIG. 1, system 100 is configured to produce syngas 140 from organic agricultural waste 111 and synthetic waste 112, in addition to methane 115 which is produced at least from livestock within the agricultural site 110. Agricultural site 110 includes livestock and other agricultural activities that generate organic agricultural waste (e.g., manure and/or rejected feed) and produce methane 115 emissions within a fully or partially enclosed structure.

The organic agricultural waste 111 is directed from the agricultural site 110 to a waste processing system 120. Synthetic waste 112, which may include materials such as feed bags and plastic wrappers, is also introduced into the waste processing system 120. Within the waste processing system 120, the organic agricultural waste 111 and synthetic waste 112 are subjected to preparatory steps such as shredding, drying, and homogenizing to produce a waste feedstock 113. This waste feedstock 113 is a mixture of processed organic and synthetic waste materials, optimized for subsequent gasification.

Methane 115 captured from the agricultural site 110 is conveyed to a gasifier 130. The methane 115 may be provided along with other gases, and may in some cases represent ambient air within one or more structures of the agricultural site 110 that contains a higher level of methane than is typically present in atmospheric air. The gasifier 130 receives both the waste feedstock 113 and methane 115, which are introduced into the gasifier 130 in a controlled manner. The gasifier 130 is configured to operate under specific conditions, including elevated temperatures and limited oxygen availability, to thermally decompose the waste feedstock 113 and methane 115 without (or with a minimal amount of) combustion. The gasification process breaks molecular bonds within the materials, resulting in the production of syngas 140.

The syngas 140, composed primarily of hydrogen and carbon monoxide, exits the gasifier 130 and can be further processed or utilized as a fuel source. System 100 integrates waste management and methane 115 capture into a single process, providing an effective method for converting agricultural and synthetic waste into syngas 140 suitable for various applications.

According to some embodiments, the agricultural site 110 may include any one or more barns or other agricultural structures that house animals. In some cases, the agricultural site 110 may house thousands of animals, such as cows, which naturally exhale methane and which is contained, at least to some extent, within the structure(s) due a lack of avenues for the methane to escape outside. A typical cow may produce between 100 and 160 kilograms of methane per year as part of its regular biological functions. The agricultural site 110 may comprise one or more fans or other devices that may be operated to circulate the air within one or more structures so that air containing methane is directed toward the gasifier and fresh air is directed into the structure(s).

In addition to methane emissions, each cow can generate approximately 1,000 kilograms of manure annually, significantly contributing to farm waste and methane emissions. Agricultural site 110 may also produces a substantial volume of mixed waste, including items such as feedbags, personal protective equipment (PPE), and plastic wrappers from test equipment or hay bales. This mixed waste can total around 136,000 kilograms per cow each year, illustrating the high volume and diversity of waste materials requiring management.

In the example of FIG. 1, organic agricultural waste 111 may include any solid and/or liquid organic waste generated by animals within the agricultural site 110. For instance, the organic agricultural waste 111 may include any one or more of: manure, crop residues, food scraps, paper, cardboard, untreated wood, and/or plant waste. In the example of FIG. 1, synthetic waste 112 may include any non-biodegradable liquid and/or solid waste generated or otherwise present within the agricultural site 110, such as plastic, metal, or treated wood.

In the example of FIG. 1, both the organic agricultural waste 111 and synthetic waste 112 are conveyed to the waste processing system 120 for conversion into waste feedstock 113. In some embodiments, either or both types of waste may be collected, at least in part, by an automated system within the agricultural site 110. For example, manure may be collected by an automated system of conveyors that collect waste from beneath animals at the site. In some cases, system 100 may automatically convey waste (e.g., by conveyors) to the waste processing system 120 from a limited number of waste collection points within the agricultural site 110 (e.g., which may be loaded manually by personnel at the site). In some embodiments, separate systems may be present within the agricultural site 110 for conveyance of the two different types of waste to the waste processing system, although in some cases a combined conveyance system may intermingle both types of waste (in which case the separate pathways of waste depicted in FIG. 1 may in fact be implemented as a single shared pipeline).

In the example of FIG. 1, the waste processing system 120 is configured to mechanically mix and/or break down received waste. In some embodiments, the waste processing system 120 comprises one or more shredders which are configured to mechanically break down waste into semi-uniform pieces. The waste processing system 120 may be configured to mechanically break down the organic agricultural waste 111 and synthetic waste 112 separately or together. In embodiments in which the waste processing system 120 is configured to mechanically break down the organic agricultural waste 111 and synthetic waste 112 separately, the broken down waste of each type may be subsequently mixed together by the waste processing system. In some embodiments, the waste processing system 120 is configured to mechanically break down only the synthetic waste prior to it being mixed with the organic agricultural waste 111 received from the agricultural site 110. For instance, the synthetic waste 112 may be shredded or otherwise mechanically broken down into smaller pieces, then mixed with manure or other organic agricultural waste 111 by the waste processing system 120.

In some embodiments, waste processing system 120 is configured to dry either or both of organic agricultural waste 111 and synthetic waste 112, such as through heating. As such, while either or both types of waste may comprise liquid, the waste processing system 120 may be configured to remove at least some of this liquid in preparing the waste feedstock 113. In some embodiments, the waste processing system 120 is configured to mix the organic agricultural waste 111 and synthetic waste 112, either or both of which may been previously heated and/or mechanically broken down by the waste processing system 120. More generally, mixing by the waste processing system 120 may be performed at any suitable step between heating and/or mechanically breaking down either or both types of waste by the waste processing system. Subsequent to mixing, and subsequent to one or more optional steps of heating and/or mechanically breaking down either or both types of waste, the resulting mixture is conveyed as waste feedstock 113 to the gasifier 130. The waste feedstock 113 may be a solid material, or in some cases may be provided as a slurry or other mixture of liquid and solid material.

In the example of FIG. 1, system 100 includes gasifier 130 configured to receive the waste feedstock 113 and methane 115 (whether provided as a component of air or otherwise) and to produce syngas 140 through non-combustion heating of the waste feedstock 113 and the methane 115. In some embodiments, system 100 comprises one or more compressors or other devices for transporting methane gas to the gasifier 130 (e.g., through compression, blowing, injection, feeding, etc.).

According to some embodiments, the gasifier may be any of a fixed-bed, moving-bed, fluidized-bed, entrained-flow, or rotary-kiln reactor system, and may incorporate an internal conveying or agitation mechanism (e.g., a screw conveyor) to advance and mix solids while maintaining an airtight environment.

In some embodiments, system 100 is configured to convey the methane 115 and the waste feedstock 113 via separate pipelines. For instance, system 100 may comprise one or more first pipelines for conveying methane gas to the gasifier 130, and one or more second pipelines for conveying the waste feedstock 113 to the gasifier. Additionally, or alternatively, system 100 may comprise a mixed-phase feed pipeline configured to convey the methane gas 115 and waste feedstock 113 together into the gasifier 130 (in which case the separate pathways of methane 115 and waste feedstock 113 depicted in FIG. 1 may in fact be implemented as a single shared mixed-phase pipeline input to the gasifier). For instance, system 100 may comprise one or more pneumatic conveyor systems for conveying a mixture of methane gas 115 and waste feedstock 113 to the gasifier.

According to some embodiments, operation of the gasifier 130 includes heating the methane 115 and the waste feedstock 113 under controlled partial-oxidation and/or pyrolysis conditions to at least partially thermally decompose the feedstock and form syngas comprising carbon monoxide and hydrogen. This controlled thermal process converts the waste into a gaseous product resembling syngas, primarily consisting of hydrogen, carbon monoxide, carbon dioxide, and traces of methane. The gasification of farm waste serves the dual purpose of reducing methane emissions and harnessing valuable gases for energy production.

For instance, the gasifier may be operated at a temperature between 700° C. and 1,500° C. and at an internal pressure between 20 bar and 40 bar. Oxygen can be present in the reactor at an oxygen equivalence ratio between 0.2 and 0.4 to favor gasification pathways while suppressing full combustion. The gasifier may be configured to accept dilute methane streams, including air from agricultural buildings containing methane between 50 ppm (parts per million) and 800 ppm, and to co-process that methane with the solid waste feedstock. In the example of FIG. 1, the gasifier 130 is configured with an outlet through which syngas 140 produced within the gasifier may be output.

In some embodiments, the gasifier housing and internals includes one or more features for solids handling (e.g., grates, screws, or fluidization distributors), syngas take-off at the upper section, and/or optional collection of carbon-rich solid residue. The system 100 may incorporate instrumentation and controls for temperature, pressure, and oxygen-equivalence monitoring to maintain targeted operating windows and to ensure stable mixed-phase feeding when implemented.

In some embodiments, excess heat generated by the gasifier 130 is utilized by system 100 to enhance system efficiency. For instance, in a combined heat and power (CHP) system, excess heat produces steam for electricity generation or industrial heating applications. Additionally, or alternatively, heat generated by the gasifier can be provided to the waste processing system 120 and used by the waste processing system to dry waste, potentially improving system efficiency by reducing input material moisture. Additionally, or alternatively, heat generated by the gasifier can be provided absorption chillers configured to convert heat into chilled water for refrigeration or air conditioning, while agricultural greenhouses can use the heat to extend growing seasons. In arid regions, the heat can even drive thermal desalination processes, converting saltwater into fresh water. These applications maximize the utility of the gasifier's byproduct heat, enhancing overall system efficiency and sustainability.

In some embodiments, the gasifier may produce one or more byproducts depending on the composition of the waste feedstock 113. The syngas 140, which is primarily hydrogen, carbon monoxide, carbon dioxide, and methane, may contain one or more of these byproducts and can be processed further. In some cases, these gases may be separated and stored, directly combusted for energy, or converted into value-added products through catalytic processes, such as transforming methane into methanol.

In some embodiments, system 100 comprises a heat recovery system configured to capture thermal energy produced by the gasifier during gasification and convert the thermal energy into electricity (e.g., to supplement the power needs of agricultural site 110). Recovered heat can also be used directly for heating or cooling applications within farm infrastructure. This process enhances energy efficiency, reduces reliance on external energy sources, and aligns with sustainable farm management practices.

In some embodiments, system 100 comprises a fuel conversion system configured to employ catalytic oxidation to transform gases like methane into valuable chemical products. For instance, using catalysts, such a fuel conversion system may convert methane into substances such as methanol, acetic acid, and ethylene gas. This process not only diversifies the range of derivable products but also enhances the economic viability of the waste-to-energy system by creating marketable chemicals for industrial use. By reducing greenhouse gas emissions and generating additional revenue streams, the catalytic process significantly improves the system's sustainability.

In some embodiments, system 100 comprises an energy storage system configured to capture and store excess energy produced by the gasifier during gasification and fuel conversion. The stored energy can be used for charging electric vehicle (EV) batteries, supplementing the local electricity grid, or providing backup power during peak demand or grid outages. This storage capability enhances the farm's energy independence and contributes to a more sustainable energy ecosystem, ensuring reliable operations even during challenging conditions.

In some embodiments, system 100 comprises a fuel and chemical storage system configured to store separated gases and/or converted products. Modular, interchangeable storage units facilitate seamless operation and continuous production cycles. Alternatively, these storage systems can be connected to natural gas pipelines or other infrastructure for immediate distribution or use. This integration streamlines the management of produced substances, supports broader energy and chemical supply chains, and strengthens the farm's role as a sustainable energy contributor.

In some embodiments, the syngas 140 may be utilized for downstream chemical conversion, such as in a Fischer-Tropsch reactor to produce liquid hydrocarbons (e.g., synthetic diesel), with catalyst selection enabling production of varied fuel fractions.

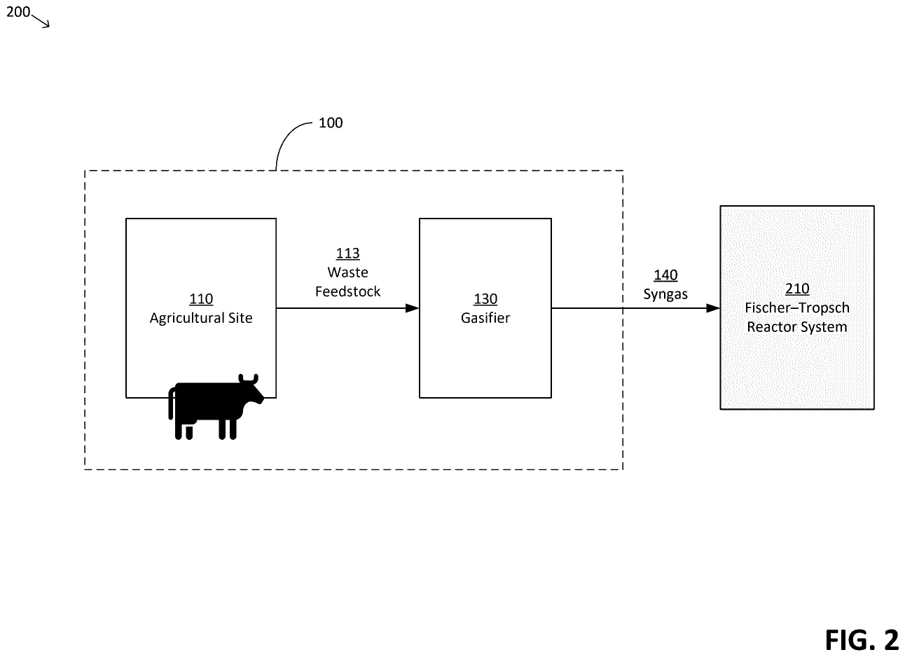

FIG. 2 depicts an illustrative example of a system suitable for performing such a process, according to some embodiments.

In the example of FIG. 2, system 100 described above produces syngas 140, which is provided to Fischer-Tropsch (FT) reactor system 210. FT reactor 210 may be configured to convert the syngas 140 into one or more liquid hydrocarbons, including synthetic diesel fuel. In some embodiments, the FT reactor 210 receives syngas comprising carbon monoxide and hydrogen and catalytically polymerizes these components into long-chain hydrocarbons and oxygenates under elevated temperature and pressure, with water and light gases formed as byproducts. In some embodiments, the FT reactor is integrated downstream of the gasifier 130 as depicted with appropriate gas cleanup and conditioning to deliver a controlled H2:CO ratio, reduced sulfur and halide content, and managed moisture levels suitable for stable FT catalysis.

In some embodiments, the FT reactor 210 is constructed as a fixed-bed tubular reactor containing supported catalysts, such as cobalt or iron on porous carriers (e.g., alumina, silica, titania), arranged to provide high surface area and uniform flow distribution. In some embodiments, the FT reactor 210 is a slurry bubble column, in which finely divided catalyst is dispersed in a liquid medium and syngas is introduced through spargers to contact the catalyst under well-mixed conditions, enabling efficient heat removal and scalable operation. In yet another implementation, the reactor may comprise a microchannel or plate-type architecture to enhance heat transfer and manage the highly exothermic FT reactions.

According to some embodiments, the FT reactor may be operated at elevated pressure, for example within the same 20-40 bar range as the upstream gasifier 130, and at moderate temperatures that favor chain growth and liquid product formation. The reactor may include internal or external heat exchangers to remove reaction heat and maintain target temperatures, catalyst baskets or retention screens to confine particulate catalysts, and distribution manifolds to provide uniform syngas delivery. Downstream product handling can include hot and cold separators to remove water and light gases, wax and heavy hydrocarbon collection systems, and fractionation units to isolate diesel-range products. By adjusting catalyst formulation and operating conditions, the reactor can be tuned to produce varied fuel fractions or specialty hydrocarbons, and may incorporate periodic regeneration or replacement procedures to maintain activity.

Integration of the FT reactor 210 with the gasifier 130 may, in some embodiments, include a syngas conditioning train comprising particulate filters, acid-gas removal units, and ratio adjustment systems to set a desired hydrogen-to-carbon monoxide ratio for FT synthesis. The FT reactor 210 can be equipped with instrumentation and controls to monitor temperature, pressure, space velocity, and product composition, thereby maintaining stable operation and consistent liquid hydrocarbon output suitable for downstream upgrading or direct use as synthetic fuels.

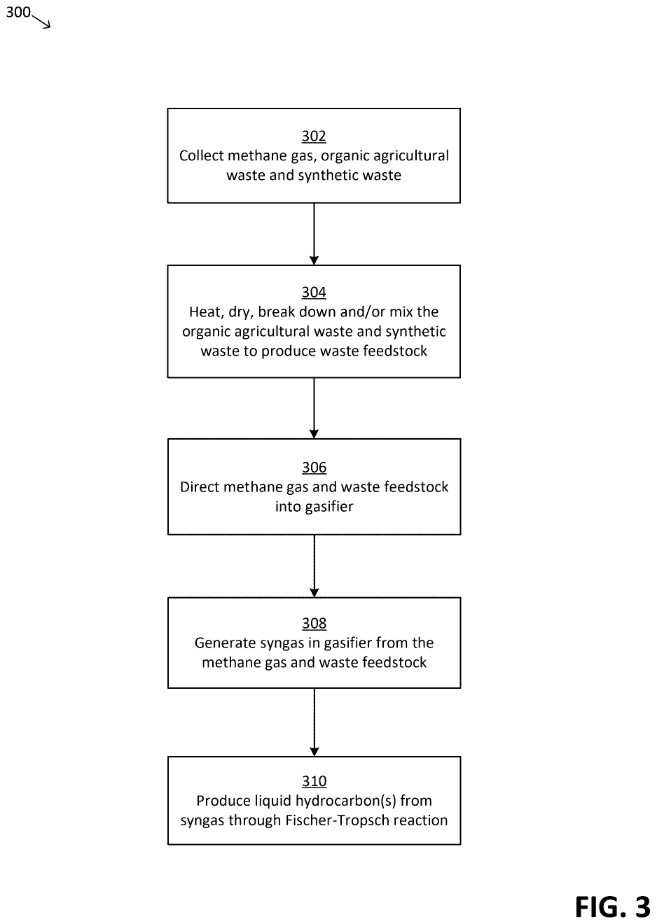

FIG. 3 is a flowchart of a methane and waste conversion method, according to some embodiments. Part or all of method 300 shown in FIG. 3 may be performed by system 100 and/or system 200 shown in FIGS. 1-2 and described above.

At step 302 of method 300, methane-containing air, organic agricultural waste, and synthetic waste are collected from an agricultural site or landfill. The organic agricultural waste may include manure, rejected feed, and other biomass-derived materials, while the synthetic waste may include plastics such as feed bags and wrappers. The methane-containing air can originate from partially enclosed agricultural buildings and may include methane at concentrations between 50 ppm and 800 ppm.

At step 304, the organic agricultural waste and synthetic waste are processed to produce a waste feedstock. Processing can include heating to drive off moisture, drying to a target moisture content suitable for gasification, mechanical size reduction such as shredding to improve handling and reaction kinetics, and mixing or homogenizing to form a consistent composite feedstock. This preparation facilitates reliable conveying and controlled thermal decomposition in the gasifier.

At step 306, the methane gas and the prepared waste feedstock are directed into a gasifier. The gasifier can be supplied via separate pipelines for gas and solids or through a mixed-phase feed pipeline configured for pneumatic conveying of gas and particulates. The introduction can occur under controlled oxygen conditions to maintain an oxygen equivalence ratio between 0.2 and 0.4.

At step 308, syngas is generated within the gasifier by heating the methane and waste feedstock under gasification or pyrolysis conditions. The reactor can operate at temperatures between 700° C. and 1,500° C. and pressures between 20 bar and 40 bar to at least partially thermally decompose the feedstock, producing a syngas comprising carbon monoxide and hydrogen. Carbon-rich solid residue may form and be withdrawn, while syngas exits through an outlet for downstream processing.

At step 310, the syngas is converted into one or more liquid hydrocarbons through a Fischer-Tropsch reaction. A downstream FT reactor receives the syngas after appropriate conditioning (e.g., particulate removal, acid-gas cleanup, and H2:CO ratio adjustment) and catalytically produces synthetic fuels, including diesel-range products, with water and light gases as byproducts. The flowchart highlights the integrated sequence from collection and preparation of mixed wastes and methane to co-gasification and catalytic synthesis of liquid hydrocarbons.

Having thus described several aspects of at least one embodiment of this disclosure, it is to be appreciated that various alterations, modifications, and improvements will readily occur to those skilled in the art. For instance, aspects of the techniques described herein may be combined in any of the following ways:
Aspect 1. A method comprising: directing methane gas and a waste feedstock into a gasifier, wherein the waste feedstock comprises organic agricultural waste and synthetic waste; and heating the methane gas and the waste feedstock in the gasifier to produce syngas.
Aspect 2. The method of aspect 1, wherein the organic agricultural waste includes manure.
Aspect 3. The method of any of aspects 1-2, further comprising mechanically breaking down the synthetic waste, and mixing broken down synthetic waste with the organic agricultural waste to produce the waste feedstock.
Aspect 4. The method of any of aspects 1-3, further comprising drying the synthetic waste prior to mixing the broken down synthetic waste with the organic agricultural waste to produce the waste feedstock.
Aspect 5. The method of any of aspects 1-4, comprising heating the methane gas and the waste feedstock in the gasifier at a temperature between 700° C. and 1,500° C.
Aspect 6. The method of any of aspects 1-5, comprising directing air from an agricultural building into the gasifier, wherein the methane gas is a component of the air from the agricultural building.
Aspect 7. The method of any of aspects 1-6, wherein the air contains the methane gas in an amount between 50 parts per million (ppm) and 800 ppm.
Aspect 8. The method of any of aspects 1-7, comprising at least partially thermally decomposing the waste feedstock in the gasifier.
Aspect 9. The method of any of aspects 1-8, wherein the syngas comprises carbon monoxide and hydrogen gas.
Aspect 10. The method of any of aspects 1-9, wherein, while heating the methane gas and the waste feedstock in the gasifier, the gasifier contains oxygen with an oxygen equivalence ratio between 0.2 and 0.4.
Aspect 11. The method of any of aspects 1-10, further comprising converting the syngas into one or more liquid hydrocarbons through a Fischer-Tropsch process.
Aspect 12. The method of any of aspects 1-11, wherein the one or more liquid hydrocarbons includes synthetic diesel fuel.
Aspect 13. The method of any of aspects 1-12, wherein directing the methane gas and the waste feedstock into the gasifier comprises directing the methane gas and the waste feedstock through a mixed-phase feed pipeline.
Aspect 14. The method of any of aspects 1-13, comprising heating the methane gas and the waste feedstock in the gasifier at an operating pressure of between 20 bar and 40 bar.
Aspect 15. A system comprising: a feed pipeline system configured to convey methane gas and a waste feedstock, wherein the waste feedstock comprises organic agricultural waste and synthetic waste; and a gasifier configured to receive the methane gas and the waste feedstock from the feed pipeline system and to heat the methane gas and the waste feedstock to produce syngas.
Aspect 16. The system of aspect 15, wherein the feed pipeline system is configured to transport the methane gas into the gasifier through one or more first pipelines and to move the waste feedstock into the gasifier through one or more second pipelines distinct from the one or more first pipelines.
Aspect 17. The system of any of aspects 15-16, wherein the feed pipeline system is configured to convey the methane gas and the waste feedstock through a mixed-phase feed pipeline.
Aspect 18. The system of any of aspects 15-17, wherein the mixed-phase feed pipeline is configured to convey the methane gas and the waste feedstock into the gasifier through pneumatic conveying.
Aspect 19. The system of any of aspects 15-18, wherein the gasifier is configured to heat the methane gas and the waste feedstock at a temperature between 700° C. and 1,500° C. and at an operating pressure between 20 bar and 40 bar.
Aspect 20. The system of any of aspects 15-19, further comprising a Fischer-Tropsch reactor configured to receive the syngas from the gasifier and to convert the syngas into one or more liquid hydrocarbons.
Aspect 21. A method for converting waste into energy using gasification, comprising: capturing methane emissions from organic sources; collecting a combination of waste materials with the methane emissions; processing the collected waste materials to create a feedstock suitable for gasification, wherein the preparation includes drying, shredding, and homogenizing the materials to achieve a consistent composition; and subjecting the prepared feedstock to a gasification process under controlled temperature and pressure conditions in the presence of a limited amount of oxygen.
Aspect 22. The method of aspect 21, further comprising utilizing the syngas produced in the gasification process as a fuel for energy production, wherein the syngas is used to generate electricity, heat, or hydrogen.
Aspect 23. The method of any of aspects 21-22, wherein the gasification process operates at a temperature between 700° C. and 1,500° C.
Aspect 24. The method of any of aspects 21-23, wherein the syngas is further processed to remove impurities to enhance its energy efficiency and suitability for end-use applications.
Aspect 25. The method of any of aspects 21-24, wherein the system includes a feedback loop to capture and recycle unconverted carbon back into the gasification process to maximize energy yield and minimize solid waste.
Aspect 26. The method of any of aspects 21-25, wherein waste heat generated during the gasification process is recovered and utilized for preheating the feedstock or generating additional energy, thereby increasing the system's overall efficiency.
Aspect 27. The method of any of aspects 21-26, wherein the captured methane is stored under pressure and blended with the prepared waste feedstock in predetermined ratios to optimize the gasification process.
Aspect 28. The method of any of aspects 21-27, further comprising integrating a control system with real-time monitoring to optimize gasification parameters based on feedstock composition and energy demand.

Such alterations, modifications, and improvements are intended to be part of this disclosure and are intended to be within the spirit and scope of the disclosure. Further, though advantages of the present disclosure are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, aspects of the disclosure may be embodied as a method, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within +5% of a target value in some embodiments, and yet within +2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially” may be used to refer to values that are within ±20% of a comparative measure in some embodiments, within ±10% in some embodiments, within ±5% in some embodiments, and yet within +2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. A method comprising:
directing methane gas and a waste feedstock into a gasifier, wherein the waste feedstock comprises organic agricultural waste and synthetic waste; and
heating the methane gas and the waste feedstock in the gasifier to produce syngas.

2. The method of claim 1, wherein the organic agricultural waste includes manure.

3. The method of claim 2, further comprising mechanically breaking down the synthetic waste, and mixing broken down synthetic waste with the organic agricultural waste to produce the waste feedstock.

4. The method of claim 3, further comprising drying the synthetic waste prior to mixing the broken down synthetic waste with the organic agricultural waste to produce the waste feedstock.

5. The method of claim 1, comprising heating the methane gas and the waste feedstock in the gasifier at a temperature between 700° C. and 1,500° C.

6. The method of claim 1, comprising directing air from an agricultural building into the gasifier, wherein the methane gas is a component of the air from the agricultural building.

7. The method of claim 6, wherein the air contains the methane gas in an amount between 50 parts per million (ppm) and 800 ppm.

8. The method of claim 1, comprising at least partially thermally decomposing the waste feedstock in the gasifier.

9. The method of claim 1, wherein the syngas comprises carbon monoxide and hydrogen gas.

10. The method of claim 1, wherein, while heating the methane gas and the waste feedstock in the gasifier, the gasifier contains oxygen with an oxygen equivalence ratio between 0.2 and 0.4.

11. The method of claim 1, further comprising converting the syngas into one or more liquid hydrocarbons through a Fischer-Tropsch process.

12. The method of claim 11, wherein the one or more liquid hydrocarbons includes synthetic diesel fuel.

13. The method of claim 1, wherein directing the methane gas and the waste feedstock into the gasifier comprises directing the methane gas and the waste feedstock through a mixed-phase feed pipeline.

14. The method of claim 1, comprising heating the methane gas and the waste feedstock in the gasifier at an operating pressure of between 20 bar and 40 bar.

15. A system comprising:
a feed pipeline system configured to convey methane gas and a waste feedstock, wherein the waste feedstock comprises organic agricultural waste and synthetic waste; and
a gasifier configured to receive the methane gas and the waste feedstock from the feed pipeline system and to heat the methane gas and the waste feedstock to produce syngas.

16. The system of claim 15, wherein the feed pipeline system is configured to transport the methane gas into the gasifier through one or more first pipelines and to move the waste feedstock into the gasifier through one or more second pipelines distinct from the one or more first pipelines.

17. The system of claim 15, wherein the feed pipeline system is configured to convey the methane gas and the waste feedstock through a mixed-phase feed pipeline.

18. The system of claim 17, wherein the mixed-phase feed pipeline is configured to convey the methane gas and the waste feedstock into the gasifier through pneumatic conveying.

19. The system of claim 15, wherein the gasifier is configured to heat the methane gas and the waste feedstock at a temperature between 700° C. and 1,500° C. and at an operating pressure between 20 bar and 40 bar.

20. The system of claim 15, further comprising a Fischer-Tropsch reactor configured to receive the syngas from the gasifier and to convert the syngas into one or more liquid hydrocarbons.
